(19) United States
(12) Patent Application Publication
(10) Pub. No.: US 2007/0211855 A1
(43) Pub. Date: Sep. 13, 2007

(54) Automatic gamma ray stereotactic radiosurgery remedial system

(76) Inventor: Xiaomu Zheng, Shenzhen (CN)

Correspondence Address: Raymond Y. Chan;Suite 128, 108 N. Ynez Avenue, Monterey Park, CA, 91754, US

(21) Appl. No.: 11/637,279

(22) Filed: Dec. 11, 2006

(30) Foreign Application Priority Data
Mar. 10, 2006 (CN) 200610034335.0

(51) Int. Cl. A61N 5/10

(52) U.S. Cl. 378/65

(57) ABSTRACT

An automatic gamma ray stereotactic radiosurgery remedial system includes an emitting source, a supporting base supporting the emitting source, and an activating mechanism driving the emitting source to move. The system further includes a treatment bed movably coupling with the supporting base, a driving mechanism driving the treatment bed to move in three dimension manner, and a head retainer upwardly extended at an end portion of the treatment bed for retaining a head of a patient so as to align the patient on the treatment bed. Therefore, the treatment bed is full-automatically moved at three directions for a focus point of the emitting source locating at each of target points of the patient for gamma ray treatment.

### BACKGROUND OF THE PRESENT INVENTION

1. Field of Invention

The present invention belonging to a medical equipment, and more particularly to an automatic gamma ray stereotactic radiosurgery remedial system, wherein an emitting source thereof is adapted to full-automatically make use of a three-dimensional coordinates system to locate the target points so as to fix the focus point at one time for radiosurgery.

2. Description of Related Arts

A conventional gamma ray y radiosurgery system comprises a head unit for emitting gamma ray for treatment. In order to operate the radiosurgery system, the operator must locate the focus point of the heat unit at the target point of the patient. Accordingly, the operator must adjust the surgery bed in a single coordinate system for locating the focus point of the heat unit at the target point of the patient lying on the surgery bed. Once the focus point of the heat unit is fixed at the target point of the patient, the operator is able to operate the radiosurgery system to emit the gamma ray towards the target point of the patient. An improved gamma ray γ radiosurgery system comprises an adjustable head unit adapted to manually adjust in a three-dimensional coordinates system so as to treat multi-target points. However, such improved gamma ray γ radiosurgery system has a major drawback that the operator must manually locate the focus point of the head unit to the target points one by one. In other words, after finishing one of the target points of the patient, the operator must move the patient out of the gamma ray γ radiosurgery system and then manually locate the focus point of the head unit to another target point in a three-dimensional coordinates system. Therefore, the frequencies of the operator moving the patient back and forth the gamma ray γ radiosurgery system is the same as the number of target points for treatment. It increases not only the time for treatment but also the pain for the patient.

### SUMMARY OF THE PRESENT INVENTION

A main object of the present invention is to provide an automatic gamma ray stereotactic radiosurgery remedial system, wherein an emitting source thereof is adapted to full-automatically make use of a three-dimensional coordinates system to locate the target points so as to fix the focus point at one time for radiosurgery.

Accordingly, in order to accomplish the above object, the present invention provides an automatic gamma ray stereotactic radiosurgery remedial system, comprising an emitting source having an emitting compartment, a supporting base supporting the emitting source, and an activating mechanism driving the emitting source to move. The automatic gamma ray stereotactic radiosurgery remedial system further comprises a treatment bed movably coupling with the supporting base, a driving mechanism driving the treatment bed to move in three dimension manner, and a head retainer upwardly extended at an end portion of the treatment bed for retaining a head of a patient so as to align the patient on the treatment bed.

According to the preferred embodiment, the driving mechanism is constructed to have three ball bearing screw shafts and three stepping motors to drive the treatment bed to move three-dimensionally. Accordingly, there are two Z-directional sliding tracks formed along two edge portions of the treatment bed at the Z-direction. There are four guiding arms parallelly extended along the Y-direction. There are two X-directional sliding tracks extended along the X-direction.

The automatic gamma ray stereotactic radiosurgery remedial system of the present invention incorporates with the X-coordinate-adjusting mechanism, a Y-coordinate-adjusting mechanism, and a Z-coordinate-adjusting mechanism to accurately move the treatment bed to move three-dimensionally. Therefore, when the patient lies on the treatment bed that the patient's head is retained at the head retainer, the treatment bed is full-automatically moved along the X, Y, and Z directions to locate the focus point at the target point of the patient.

### BRIEF DESCRIPTION OF THE DRAWINGS

FIG. 1 is a sectional view of an automatic gamma ray stereotactic radiosurgery remedial system according to a preferred embodiment of the present invention.

FIG. 2 illustrates a mechanism of the automatic gamma ray stereotactic radiosurgery remedial system according to the above preferred embodiment of the present invention.

FIG. 3 is sectional view of the treatment bed of the automatic gamma ray stereotactic radiosurgery remedial system according to the above preferred embodiment of the present invention.

### DETAILED DESCRIPTION OF THE PREFERRED EMBODIMENT

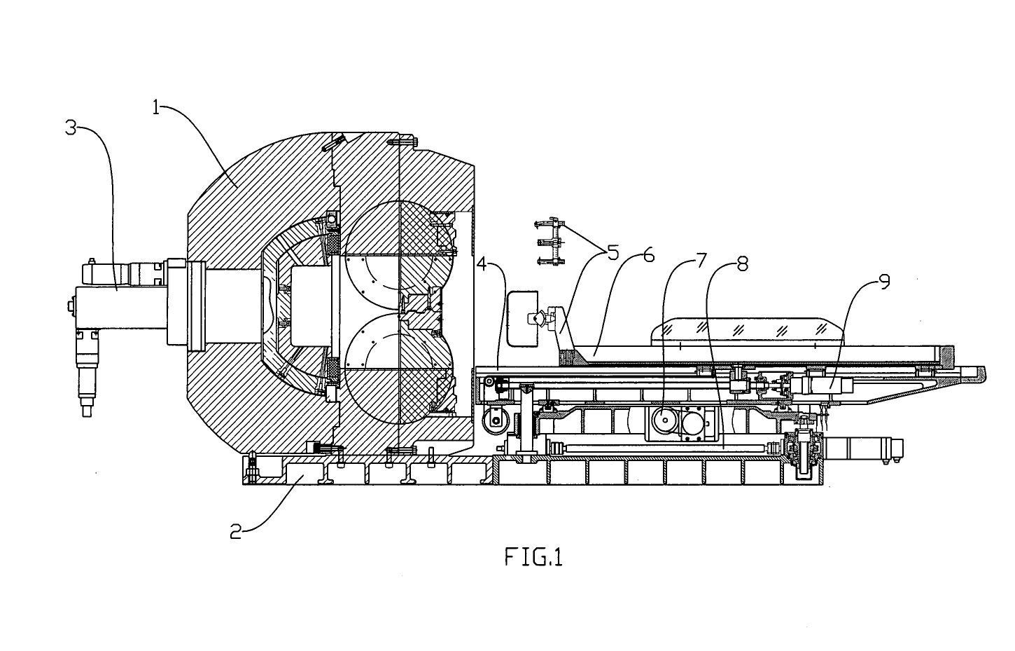

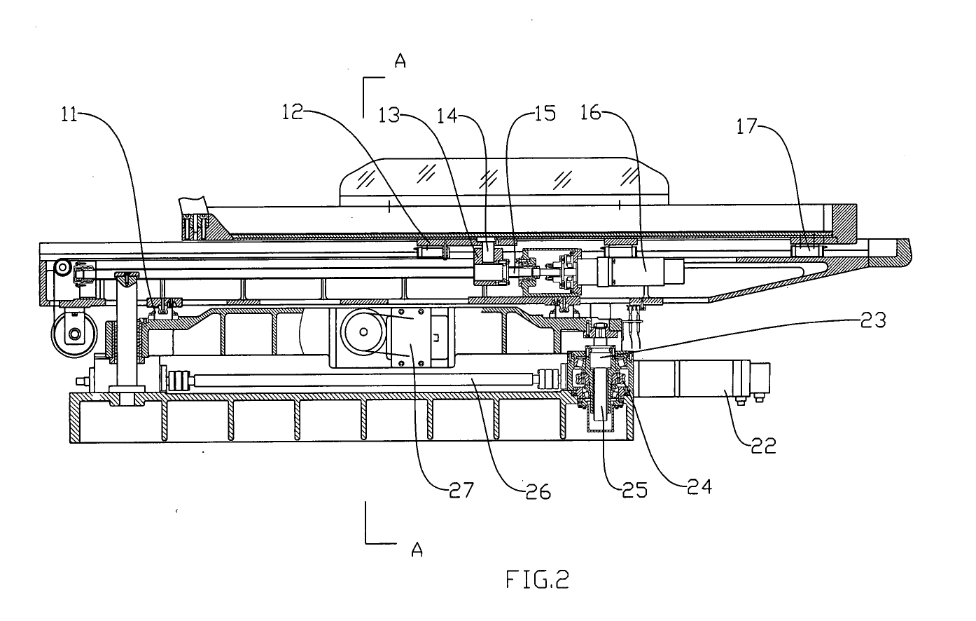

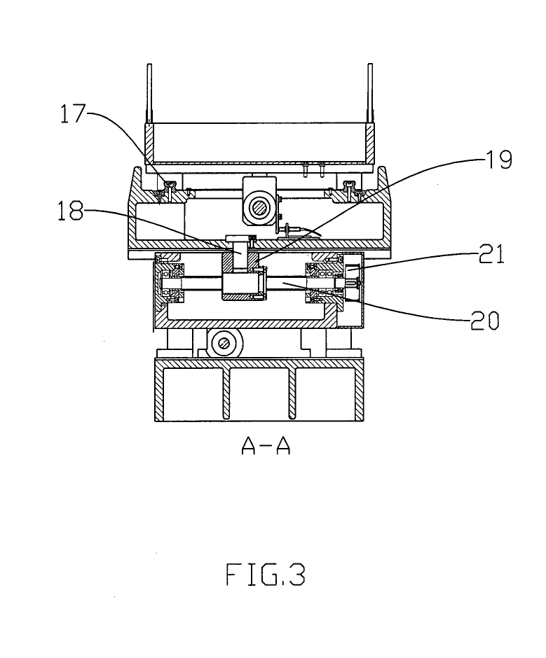

Referring to FIGS. 1 to 3 of the drawings, an automatic gamma ray stereotactic radiosurgery remedial system according to a preferred embodiment of the present invention is illustrated, wherein the automatic gamma ray stereotactic radiosurgery remedial system comprises an emitting source 1 having an emitting compartment, a supporting base 2 supporting the emitting source 1, an activating mechanism 3 movably driving the emitting source 1 to move, a treatment bed 4 movably coupling with the supporting base 2, a driving mechanism driving the treatment bed 4 to move in three dimension manner, and a head retainer 5 upwardly extended at an end portion of the treatment bed 4 for retaining a head of a patient so as to align the patient on the treatment bed 4.

According to the preferred embodiment, the operator does not require to manually control the movement of the treatment bed 4 for gamma ray treatment. The treatment bed 4 is full-automatically moved in X, Y, and Z coordinates for the treatment. Accordingly, the driving mechanism comprises a X-coordinate-adjusting mechanism 7, a Y-coordinate-adjusting mechanism 8, and a Z-coordinate-adjusting mechanism 9 operatively and individually coupling with the treatment bed 4 so as to move the treatment bed 4 in a three-dimensional manner. The X-coordinate-adjusting mechanism 7, the Y-coordinate-adjusting mechanism 8, and the Z-coordinate-adjusting mechanism 9 are constructed to form three ball bearing screw shafts and three stepping motors to drive the ball bearing screw shafts to rotate, so as to three-dimensional move the treatment bed 4 at X, Y, and Z directions. It is worth to mention that the treatment bed 4 is adjustably moved that the movement thereof has an accuracy of 0.01 mm. In other words, the error of the three-dimensional movement of the treatment bed 4 is less than 0.01 mm, such that the focus point of the emitting source can be accurately locate at the target point of the patient. In other words, when the head of the user is retained at the head retainer 5, the present invention is adapted to full-automatically locate the target points at the head of the user so as to fix the focus point at one time for radiosurgery.

The principle and process of the present invention are shown as follows:

There are two Z-directional sliding tracks 17 extended under the treatment bed 4 along two edge portions thereof at the Z-direction to guide the treatment bed 4 moving at the Z-direction. There are four guiding arms 27 parallelly extended under the treatment bed 4 along the Y-direction to guide the treatment bed 4 moving at the Y-direction. There are two X-directional sliding tracks 11 extended under the treatment bed 4 along the X-direction to guide the treatment bed 4 moving at the X-direction. Accordingly, the Z-directional sliding tracks 17, the guiding arms 27, and the X-directional sliding tracks are extended perpendicularly with each other.

In responsive to the diagnostic of the treatment, the control center sends out a Z-directional control signal to the motor 16 to drive the ball-bearing screw shaft 15 to rotate. Then, the ball-bearing screw shaft 15 drives a nut base 13 to move a bed top 12 of the treatment bed 4 in a Z-direction through a transmission element 14.

In responsive to the diagnostic of the treatment, the control center sends out a Y-directional control signal to the motor 22 to drive the ball-bearing nut shaft 24 to rotate via the worm gear 23. Then, the ball-bearing nut shaft 24 drives the ball-bearing screw shaft 25 to move so as to move the bed top 12 of the treatment bed 4 in a Y-direction. In order to ensure the stabilization of the bed top 12 and the even distribution of the transmitting force to the bed top 12 during the movement thereof along the Y-direction, each of the worm gear 23, the ball-bearing nut shaft 24, and the ball-bearing screw shaft 25 is formed in pairs. In other words, there are two worm gears 23, two ball-bearing nut shafts 24, and two ball-bearing screw shafts 25 for transmitting the force from the motor 22 via the transmission shaft 26.

In responsive to the diagnostic of the treatment, the control center sends out a X-directional control signal to the motor 27 to drive the ball-bearing screw shaft 20 through a belt-wheel 21 via an endless transmission belt. Then, the ball-bearing screw shaft 20 drives a nut base 19 to move a bed top 12 of the treatment bed 4 at the X-direction through a transmission element 18. Therefore, according to the X-directional, Y-directional, and Z-directional control signals from the control center, the treatment bed 4 is adapted to be selectively and individually moved along the X, Y, and Z directions. It is worth to mention that the treatment bed 4 can be automatically and controllably moved along the X, Y, and Z directions at the same time while being time effective. Therefore, the operator does not need to manually move the treatment bed 4 along the X, Y, and Z directions. Importantly, once the target points of the patient are set, the automatic gamma ray stereotactic radiosurgery remedial system of the present invention is programmed to automatically locate the focus point of the emitting source 1 at each of the target points of the patient such that after one of the target points of the patient is treated, the treatment bed 4 is automatically moved along the X, Y, and Z directions to locate the focus point of the emitting source 1 at another target point of the patient so as to subsequently emit the gamma ray to all the target points of the patient at one continuous time.

What is claimed is:

1. An automatic gamma ray stereotactic radiosurgery remedial system, comprising:
an emitting source;
a supporting base supporting said emitting source;
an activating mechanism driving said emitting source to move;
a treatment bed movably coupling with said supporting base;
a driving mechanism driving said treatment bed to move in a three-dimension manner; and
a head retainer upwardly extended at an end portion of said treatment bed for retaining a head of a patient so as to align said patient on said treatment bed, wherein said treatment bed is full-automatically moved at three directions for a focus point of said emitting source locating at each of target points of said patient for gamma ray treatment.

2. The automatic gamma ray stereotactic radiosurgery remedial system, as recited in claim 1, wherein said driving mechanism comprises X-direction controlling means for controllably moving said treatment bed at X-direction, Y-direction controlling means for controllably moving said treatment bed at Y-direction, Z-direction controlling means for controllably moving said treatment bed at Z-direction, and a control center controlling an operation of each of said means.

3. The automatic gamma ray stereotactic radiosurgery remedial system, as recited in claim 2, wherein said X-direction controlling means comprises a first motor, a first ball-bearing screw shaft driven be said first motor to rotate through a belt-wheel, and a X-direction nut base driven by said first ball-bearing screw shaft to move said treatment bed at a X-direction.

4. The automatic gamma ray stereotactic radiosurgery remedial system, as recited in claim 3, wherein said Y-direction controlling means comprises a second motor, a second ball-bearing screw shaft driven by said second motor to rotate through a worm gear, and a ball-bearing nut shaft driven by said second ball-bearing screw shaft to move said treatment bed at a Y-direction.

5. The automatic gamma ray stereotactic radiosurgery remedial system, as recited in claim 4, wherein said Z-direction controlling means comprises a third motor, a third ball-bearing screw shaft driven by said third motor to rotate, and a Z-direction nut base driven by said third ball-bearing screw shaft to move said treatment bed at a Y-direction.

6. The automatic gamma ray stereotactic radiosurgery remedial system, as recited in claim 1, wherein said treatment bed comprises a bed top for supporting said patient thereon, wherein said driving mechanism are operatively coupling with said bed top to move said bed top to move at X, Y, and Z directions.

7. The automatic gamma ray stereotactic radiosurgery remedial system, as recited in claim 2, wherein said treatment bed comprises a bed top for supporting said patient thereon, wherein said X-direction controlling means, said Y-direction controlling means, and said Z-direction controlling means are operatively coupling with said bed top to move said bed top to move at X, Y, and Z directions.

8. The automatic gamma ray stereotactic radiosurgery remedial system, as recited in claim 5, wherein said treatment bed comprises a bed top for supporting said patient thereon, wherein said X-direction controlling means, said Y-direction controlling means, and said Z-direction controlling means are operatively coupling with said bed top to move said bed top to move at X, Y, and Z directions.

9. The automatic gamma ray stereotactic radiosurgery remedial system, as recited in claim 2, wherein said control center is programmed to selectively send out a X-directional control signal, a Y-directional control signal, and Z-directional control signal to said X-direction controlling means, said Y-direction controlling means, and said Z-direction controlling means respectively so as to control said treatment bed to move at X, Y, and Z directions individually.

10. The automatic gamma ray stereotactic radiosurgery remedial system, as recited in claim 5, wherein said control center is programmed to selectively send out a X-directional control signal, a Y-directional control signal, and Z-directional control signal to said X-direction controlling means, said Y-direction controlling means, and said Z-direction controlling means respectively so as to control said treatment bed to move at X, Y, and Z directions individually.

11. The automatic gamma ray stereotactic radiosurgery remedial system, as recited in claim 8, wherein said control center is programmed to selectively send out a X-directional control signal, a Y-directional control signal, and Z-directional control signal to said X-direction controlling means, said Y-direction controlling means, and said Z-direction controlling means respectively so as to control said treatment bed to move at X, Y, and Z directions individually.

12. The automatic gamma ray stereotactic radiosurgery remedial system, as recited in claim 5, wherein said first, second, and third motors are three individual stepping motors to drive said treatment bed to move at X, Y, and Z directions individually.

13. The automatic gamma ray stereotactic radiosurgery remedial system, as recited in claim 8, wherein said first, second, and third motors are three individual stepping motors to drive said treatment bed to move at X, Y, and Z directions individually.

14. The automatic gamma ray stereotactic radiosurgery remedial system, as recited in claim 11, wherein said first, second, and third motors are three individual stepping motors to drive said treatment bed to move at X, Y, and Z directions individually.

15. The automatic gamma ray stereotactic radiosurgery remedial system, as recited in claim 5, wherein said X-direction controlling means further comprises two X-directional sliding tracks extended under said treatment bed along the X-direction to guide said treatment bed moving at the X-direction, wherein said Y-direction controlling means further comprises four guiding arms parallelly extended under said treatment bed along the Y-direction to guide said treatment bed moving at the Y-direction, wherein and said Z-direction controlling means two Z-directional sliding tracks extended under said treatment bed along two edge portions thereof at the Z-direction to guide said treatment bed moving at the Z-direction.

16. The automatic gamma ray stereotactic radiosurgery remedial system, as recited in claim 8, wherein said X-direction controlling means further comprises two X-directional sliding tracks extended under said treatment bed along the X-direction to guide said treatment bed moving at the X-direction, wherein said Y-direction controlling means further comprises four guiding arms parallelly extended under said treatment bed along the Y-direction to guide said treatment bed moving at the Y-direction, wherein and said Z-direction controlling means two Z-directional sliding tracks extended under said treatment bed along two edge portions thereof at the Z-direction to guide said treatment bed moving at the Z-direction.

17. The automatic gamma ray stereotactic radiosurgery remedial system, as recited in claim 14, wherein said X-direction controlling means further comprises two X-directional sliding tracks extended under said treatment bed along the X-direction to guide said treatment bed moving at the X-direction, wherein said Y-direction controlling means further comprises four guiding arms parallelly extended under said treatment bed along the Y-direction to guide said treatment bed moving at the Y-direction, wherein and said Z-direction controlling means two Z-directional sliding tracks extended under said treatment bed along two edge portions thereof at the Z-direction to guide said treatment bed moving at the Z-direction.

18. The automatic gamma ray stereotactic radiosurgery remedial system, as recited in claim 15, wherein said Z-directional sliding tracks, said guiding arms, and said X-directional sliding tracks are extended perpendicularly with each other.

19. The automatic gamma ray stereotactic radiosurgery remedial system, as recited in claim 16, wherein said Z-directional sliding tracks, said guiding arms, and said X-directional sliding tracks are extended perpendicularly with each other.

20. The automatic gamma ray stereotactic radiosurgery remedial system, as recited in claim 17, wherein said Z-directional sliding tracks, said guiding arms, and said X-directional sliding tracks are extended perpendicularly with each other.
